(19) United States
(12) Patent Application Publication
(10) Pub. No.: US 2008/0286281 A1
(43) Pub. Date: Nov. 20, 2008

(54) CD25 BINDING MOLECULES FOR THE USE IN THE TREATMENT OF MANIFESTATIONS OF REJECTION IN TRANSPLANTATION

(76) Inventors: Gilles Feutren, Mulhouse (FR); Richard Kimberley Howell, Cheshire (GB); Peter Marbach, Therwil (CH); Andrew Roberts, West Sussex (GB); Max H. Schreier, Basel (CH); Karin Schreier, Basel (CH); Daniel Mark Schreier, Basel (CH); Anja Bettina Schreier, Basel (CH); Manfred Schulz, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/180,660

(22) Filed: Jul. 28, 2008

Related U.S. Application Data
(63) Continuation of application No. 09/862,212, filed on May 22, 2001.
Application No. 09/862,212 is a continuation of application No. PCT/EP99/08988, filed on Nov. 22, 1999.

(30) Foreign Application Priority Data
Nov. 23, 1998 (GB) 9825632.4

(51) Int. Cl. A61K 39/395; C07K 16/00; A61P 37/06

(52) U.S. Cl. 424/143.1; 530/388.1

(57) ABSTRACT

Administration of monoclonal antibodies specific for IL-2R to xenograft recipients, or long-term to immunosuppression-intolerant or non-compliant patients over a period of time beyond the very early phase of organ transplantation, prevents transplant rejection.

The invention is directed to the use of a CD25 binding molecule in the treatment of rejection in transplantation.

A serious problem following transplantation is associated with the fact that transplant patients receive a long-term immunosuppression, a so-called triple therapy composed of a calcineurin inhibitor like cyclosporin A or FK-506, a steroid and a concomitant immunosuppressant like azathioprine, mycophenolic acid, mycophenolate mofetil, a 15-deoxyspergualine, rapamycin or 40-O-(2-hydroxy)ethyl-rapamycin (RAD001). Some patients, in particular juvenile and adolescent patients, however, have difficulties in accepting the constraints of the treatment and show poor compliance. Others are intolerant to calcineurin inhibitors, and develop severe adverse side effects, e.g. renal disfunction, hirsutism, gingival hyperplasia and hypertension. As a result many patients decrease the dosage of the respective medication or stop it entirely. The result for both groups is an unsatisfactory level of immunosuppression with the threat of losing the transplant.

Monoclonal antibodies specific for the interleukin-2 receptor (IL-2R) are currently used for the so-called induction treatment, i.e. as prophylactic short-term immunosuppressants for single or multiple administration in the very early phase following transplantation, e.g. shortly before the transplantation and up to 3 months after transplantation.

Surprisingly, H has now been discovered that the administration of monoclonal antibodies specific for IL-2R over a period of time beyond the very early phase following transplantation to immunosuppression-intolerant or -non-compliant patients decreases the risk of trans-plant rejection resulting from a reduced level of immunosuppression, especially from reduced levels of calcineurin inhibition.

A period of time beyond the very early phase following transplantation is that period of time that begins some weeks, months or even years after transplantation depending on the nature of the side effects and on the patient's perception of the severity of the side effects, and may last for the rest of life of the transplant recipient. The reduction in calcineurin inhibitor dose may occur in the first weeks because of immediate side effects, or, especially in pediatric patients, much later as the patient matures and becomes aware of cosmetic changes that can effect social behaviour.

Compounds suitable for the purposes of this invention include monoclonal antibodies specific for the α subunit of IL-2R (≅55 kDa), CD25, i.e. IL-2Rα, e.g. a humanized antibody, e.g. a humanized antibody having two pairs of light/heavy chain dimers, wherein each chain comprises complementarity determining regions (CDR's) and human-like framework regions, wherein the CDR's are from different immunoglobulin molecules than the framework regions, or a chimeric antibody, e.g. a chimeric antibody which comprises at least one antigen binding site comprising at least one domain which comprises in sequence, the hypervariable regions CDR1, CDR2 and CDR3; said CDR1 having the amino add sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe; or direct equivalents thereof.

Humanized antibodies have been disclosed together with processes for their preparation in WO 90/07861, the content of which is incorporated herein by reference. These antibodies have, on the basis of observed activity in e.g. a test to determine antibody-dependent cell mediated cytotoxicity, been found to be useful as immunosuppressant for the prevention of graft rejection. An individual antibody of WO 90/07861, daclizumab, is multiply administered to transplant patients during the first three months following transplantation.

Chimeric antibodies have been disclosed together with processes for their preparation in EP 449,769, the content of which is incorporated herein by reference. These antibodies have, on the basis of observed activity in e.g. a test to determine cell proliferation, been found to be useful as immunosuppressant for the prevention of graft rejection. An individual antibody of EP 449,769, basiliximab, is twice administered to transplant patients on the day of trans-plantation and 4 days later.

Individual antibodies suitable for use in accordance with the present invention are the humanized antibody daclizumab and the chimeric antibody basiliximab.

A preferred compound for use in accordance with the present invention is basiliximab which is commercially available as SIMULECT® from Novartis.

In accordance with the particular findings of the present invention, there is provided:

1. A method of preventing or treating transplant rejection in a recipient of organ, tissue or unmodified or modified cell transplant, e.g. heart, lung, combined heart-lung, trachea, liver, kidney, pancreas, Islet cell, bowel, e.g. small bowel, skin, muscles or limb, bone marrow, esophagus, cornea or nervous tissue transplant, which method comprises long-term administering to said recipient an effective amount of a monoclonal antibody specific for IL-2R, e.g. a humanized or chimeric monoclonal antibody specific for IL-2R, especially basiliximab or daclizumab, over a period of time beyond the very early phase following transplantation.

In a preferred embodiment the invention provides

2. A method of preventing or treating rejection in an immunosuppression-intolerant or -non-compliant recipient of organ, tissue or unmodified or modified cell transplant, e.g. heart, lung, combined heart-lung, trachea, liver, kidney, pancreas, Islet cell, bowel, e.g. small bowel, skin, muscles or limb, bone marrow, esophagus, cornea or nervous tissue transplant, which method comprises long-term administering to said recipient an effective amount of a monoclonal antibody specific for IL-2R, e.g. a humanized or chimeric mono clonal antibody specific for IL-2R, especially basiliximab or daclizumab, over a period of time beyond the very early phase following transplantation.

The organ, tissue or unmodified or modified cell transplant may be of human (allotransplantation) or non-human, e.g. pig, (xenotransplantation) origin.

In further embodiments, the present invention provides:

3.1 A method of preventing or treating xenotransplant rejection in a recipient of an organ, tissue or unmodified or modified cell xenotransplant, e.g. heart, lung, combined heart-lung, trachea, liver, kidney, pancreas, Islet cell, bowel, e.g. small bowel, skin, muscles or limb, bone marrow, esophagus, cornea or nervous tissue transplant which method comprises administering to said recipient an effective amount of a monoclonal antibody specific for IL-2R, e.g. a humanized or chimeric monoclonal antibody specific for IL-2R, especially basiliximab or daclizumab.

3.2 A method of preventing or treating rejection in a recipient of an organ, tissue or unmodified or modified cell xenotransplant, e.g. heart, lung, combined heart-lung, trachea, liver, kidney, pancreas, Islet cell, bowel, e.g. small bowel, skin, muscles or limb, bone marrow, esophagus, cornea or nervous tissue transplant, which method comprises administering to said recipient an effective amount of a monoclonal antibody specific for IL-2R, e.g. a humanized or chimeric monoclonal antibody specific for IL-2R, especially basiliximab or daclizumab.

As alternative to the above the present invention also provides:

4. A monoclonal antibody specific for IL-2R, e.g. a humanized or chimeric monoclonal anti-body specific for IL-2R, especially basiliximab or daclizumab, for use in any method as defined under 1 to 3 above; or

5. A monoclonal antibody specific for IL-2R, e.g. a humanized or chimeric monoclonal anti-body specific for IL-2R, especially basiliximab or daclizumab, for use in the preparation of a pharmaceutical composition for use in any method as defined under 1 to 3 above; or

6. A pharmaceutical composition for use in any method as defined under 1 to 3 above comprising a monoclonal antibody specific for IL-2R, e.g. a humanized or chimeric monoclonal antibody specific for IL-2R, especially basiliximab or daclizumab, together with one or more pharmaceutically acceptable diluents or carriers therefor.

Utility of a monoclonal antibody specific for IL-2R, e.g. a humanized or chimeric monoclonal antibody specific for IL-2R, especially basiliximab or daclizumab, in preventing transplant rejection as hereinabove specified, may be demonstrated in clinic where e.g. the trans-planted organ, tissue or unmodified or modified cell transplant may be submitted to regular controls, e.g. to biopsy controls or ultrasound scanning. Daclizumab may be useful as rescue treatment when the first symptoms of rejection occur.

### I. Partial Replacement of Calcineurin Inhibitor by Monoclonal Antibody Specific for IL-2R

30 patients intolerant to cyclosporine A are randomized to one of two treatment groups; those receiving a maintenance dose of Sandimmun Neoral® sufficient to achieve cyclosporine A trough levels above 200 ng/ml and those receiving a maintenance dose of Sandimmun Neoral® sufficient to achieve cyclosporine A trough levels above 100 ng/ml+ a monoclonal antibody specific for IL-2R, e.g. basiliximab or daclizumab. The patients are also on stable doses of prednisone. The monoclonal antibody specific for IL-2R is administered intravenously at a dose of 40 mg every month.

During the treatment, markers of rejection, e.g. rise in creatinine or decreased GFR (for renal transplant recipients) or rising liver transaminases (for liver transplant recipients), and of calcineurin inhibitor intolerance, e.g. renal dysfunction, hirsutism, hypertension, gingival and hyperplasia, are monitored.

When a calcineurin inhibitor intolerant patient receives a monoclonal antibody specific for IL-2R, e.g. a humanized or chimeric monoclonal antibody specific for IL-2R, especially basiliximab or daclizumab, and a reduced dosage of a calcineurin inhibitor, e.g. cyclosporine A, side effects of the calcineurin inhibitor are reduced. Patients receiving the lower dose of Sandimmun Neoral® + a monoclonal antibody specific for IL-2R compared with those receiving the higher dose of Sandimmun Neoral® and no monoclonal antibody specific for IL-2R show a satisfactory level of immunosuppression. The monoclonal antibody specific for IL-2R, e.g. basiliximab or daclizumab is effective when administered at a dose of 40 mg every month.

The beneficial effects of a monoclonal antibody specific for IL-2R, e.g. basiliximab or daclizumab, in xenotransplantation may be tested clinically.

The use of a monoclonal antibody specific for IL-2R according to the invention results in a better outcome than the as yet available therapies.

The appropriate dosage will, of course, vary depending upon, for example, the particular molecule to be employed, the host, the mode of administration and the severity of the condition being treated and the effects obtained. Satisfactory results are generally indicated to be obtained at dosages in the range of from about 0.1 mg to about 500 mg, e.g. of from 10 mg to 150 mg, especially 40 mg or 20 mg.

For preventing or treating rejection in an immunosuppression-intolerant or -non-compliant recipient of organ, tissue or unmodified or modified cell transplant, administration of a dose in the range of from 10 to 150 mg, especially 40 mg, may be on a weekly or monthly basis, for example every week, every two, three, four, five, six, seven or eight weeks, regularly or irregularly, as required.

An exemplary dosing regimen for preventing or treating rejection in an immunosuppression-intolerant or -non-compliant recipient of organ, tissue or unmodified or modified cell trans-plant beyond the very early phase following transplantation, is long-term monthly intravenous administration of 40 mg.

For preventing or treating rejection in a xenograft transplantation recipient, administration of a dose in the range of from 10 to 150 mg, especially 20 mg, may be within a day prior to transplantation followed by a single dose in the range of from 10 to 150 mg, especially 20 mg, within a week following transplantation or by one to 10 doses in the range of from 10 to 150 mg, especially 20 mg, for up to 10 weeks following transplantation; optionally followed by administration as often as indicated on a weekly or monthly basis, for example a dose in the range of from 10 to 150 mg, especially 40 mg, may be administered every week, every two, three, four, five, six, seven or eight weeks, regularly or irregularly, as required.

An exemplary dosing regimen for preventing rejection in a xenograft transplantation recipient is intravenous administration of 20 mg two hours prior to transplantation and 20 mg 4 days later; or 20 mg two hours prior to transplantation, 20 mg 4 days later, followed by monthly intravenous administration of 40 mg. Another exemplary dosing regimen for pre-venting rejection in a xenograft transplantation recipient is intravenous administration of 5 times 1 mg/kg body weight over 8 weeks; or intravenous administration of 1 mg/kg body weight every 10 days; or 5 times 1 mg/kg body weight over 8 weeks, followed by monthly intravenous administration of 2 mg/kg body weight.

Pharmaceutical compositions of the invention may be manufactured in a conventional manner as described, e.g. in EP 449769 or WO 90/07861. The composition may e.g. be prepared for storage as lyophilized powder in a vial. The solution for parenteral administration may conveniently be prepared shortly before administration.

The monoclonal antibodies specific for IL-2R may be administered together with other drugs in immunomodulating regimens or other anti-inflammatory agents. The monoclonal anti-bodies specific for IL-2R may be used in combination with cyclosporins, rapamycins or ascomycins, or their immunosuppressive analogs, e.g. cyclosporin A, cyclosporin G, FK-506, RAD001, rapamycin etc.; corticosteroids; cyclophosphamide; azathioprine; methotrexate; brequinar; leflunomide; mizoribine; mycophenolic acid; mycophenolate mofetil; 15-deoxyspergualine; other immuno-suppressive monoclonal antibodies, e.g. monoclonal anti-bodies to leukocyte receptors, e.g. MHC, CD2, CD3, CD4, CD7, CD28, B7, CD40, CD45, or CD58 or their ligands; or other immunomodulatory compounds, e.g. CTLA41g. Murine monoclonal antibodies suitable for use together with the monoclonal antibody are described in EP 449,769.

Dosages of the co-administered immunosuppressant or immunomodulatory compounds will of course vary depending on the type of co-drug employed, e.g. whether it is a steroid or a cyclosporin, on the specific drug employed, on the condition being treated, and so forth. In accordance with the foregoing the present invention provides in a yet further aspect:

7. A method as defined above comprising co-administration, e.g. concomitantly or in sequence, of a therapeutically effective amount of a monoclonal antibody specific for IL-2R and a second drug substance, said second drug substance being an immunosuppressant or immunomodulatory drug, e.g. as indicated above.

If the monoclonal antibody is co-administered with a further drug substance both may be packaged separately within the same container, with instructions for mixing or concomitant administration. Examples of kits include for example a multi-barreled syringe or a twin pack containing separate unit dose forms.

Investigations so far indicate that the administration of the monoclonal antibody is free from unacceptable side-effects at the dosage levels employed. Particularly the preferred one, basiliximab, is safe, approved by the Federal Drug Administration (FDA) of the United States and is commercially available.

1. A method of preventing or treating transplant rejection in a recipient of organ, tissue or unmodified or modified cell transplant, which method comprises long-term administering to said recipient an effective amount of a monoclonal antibody specific for IL-2R, over a period of time beyond the very early phase following transplantation.

2. A method of preventing or treating rejection in an immunosuppression-intolerant or -non-compliant recipient of organ, tissue or unmodified or modified cell transplant, which method comprises long-term administering to said recipient an effective amount of a monoclonal antibody specific for IL-2R, over a period of time beyond the very early phase following transplantation.

3. A method of preventing or treating xenotransplant rejection in a recipient of an organ, tissue or unmodified or modified cell xenotransplant, which method comprises administering to said recipient an effective amount of a monoclonal antibody specific for IL-2R.

4. (canceled)

5. A monoclonal antibody specific for IL-2R for use in a method according to claim 1.

6. A monoclonal antibody specific for IL-2R for use in the preparation of a pharmaceutical composition for use in a method according to claim 1.

7. A pharmaceutical composition for use in a method according to claim 1 comprising a monoclonal antibody specific for IL-2R together with one or more pharmaceutically acceptable diluents or carriers therefor.

8. A method according to claim 1 comprising co-administration of a therapeutically effective amount of a monoclonal antibody

8. A method according to claim 1 comprising co-administration of a therapeutically effective amount of a monoclonal antibody specific for IL-2R and a second drug substance, stance, said second drug substance being an immunosuppressant or immunomodulatory drug, e.g. as indicated above.
